(19) United States
(12) Patent Application Publication
(10) Pub. No.: US 2024/0003880 A1
(43) Pub. Date: Jan. 4, 2024

(54) MONOCLONAL ANTIBODIES AGAINST SARS-COV-2 NUCLEOCAPSID PHOSPHOPROTEIN AND SANDWICH ELISA METHOD

(71) Applicant: The Trustees of Columbia University in the City of New York, New York, NY (US)

(72) Inventors: David D. HO, Bedford, NY (US); Lihong LIU, Shanghai (CN)

(21) Appl. No.: 18/005,581

(22) PCT Filed: Jul. 16, 2021
(86) PCT No.: PCT/US2021/041955

§ 371 Date: Jan. 13, 2023

Related U.S. Application Data
(60) Provisional application No. 63/058,751, filed on Jul. 30, 2020.
(60) Provisional application No. 63/053,112, filed on Jul. 17, 2020.

(51) Int. Cl. G01N 33/569; C07K 16/10

(52) U.S. Cl. CPC G01N 33/56983; C07K 16/1003; G01N 2333/165; C07K 2317/92; G01N 2470/04; C07K 2317/21; G01N 2469/10

(57) ABSTRACT

Disclosed herein is a kit for detecting or quantifying a SARS-CoV-2 nucleocapsid phosphoprotein, including a first antibody, wherein a variable heavy chain domain comprises the amino acid sequence selected from the group consisting of SEQ ID NOs: 1, 3, 5, 7, 11, 13, 15, 17, 19, and 21, and a variable light chain domain comprises the amino acid sequence selected from the group consisting of SEQ ID NOs: 2, 4, 6, 8, 12, 14, 16, 18, 20, and 22; and a second antibody, wherein a variable heavy chain domain comprises the amino acid sequence selected from the group consisting of SEQ ID NOs: 9 and 13, and a variable light chain domain comprises the amino acid sequence selected from the group consisting of SEQ ID NOs: 10 and 14.

This application claims the benefit of U.S. Provisional Application No. 63/058,751, filed Jul. 30, 2020 and U.S. Provisional Application No. 63/053,112, filed Jul. 17, 2020, the contents of all of which are incorporated by reference.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### TECHNICAL FIELD

The present invention relates generally to monoclonal antibodies against SARS-CoV-2 nucleocapsid phosphoprotein. More particularly, the present invention relates to systems and methods for sandwich (or capture) enzyme-linked immunosorbent assays (ELISAs) for the detection of SARS-CoV-2 nucleocapsid phosphoprotein antigens.

### BACKGROUND

A novel coronavirus emerged in December 2019 in Wuhan, China and devasted Hubei Province in early 2020 before spreading to every province within China and every country in the world. This pathogen, now termed severe acute respiratory syndrome coronavirus 2 (SARS-CoV-2), has caused a global pandemic, with ˜12.5 million cases and ˜550,000 deaths reported through Jul. 10, 2020. The disease caused by SARS-CoV-2 infection is called coronavirus disease 2019 (COVID-19).

Testing by polymerase chain reaction (PCR) has been the mainstay for confirming SARS-CoV-2 infection worldwide. However, PCR is an inadequate diagnostic tool because it is complex and slow to perform and analyze. PCR tests also suffer from a high false negative rate which has been estimated to be about 20%. See Xiao, A. T., et al., False-negative of RT-PCR and Prolonged Nucleic Acid Conversion in COVID-19: Rather than Recurrence. Journal of Medical Virology (2020). The U.S. Food and Drug Administration (FDA) has approved the Sofia 2 SARS Antigen FIA test which is an immunofluorescent sandwich assay intended for the qualitative detection of the nucleocapsid protein antigen from SARS-CoV-2 in nasopharyngeal and nasal swab specimens directly or after the swabs have been added to viral transport media from individuals who are suspected of COVID-19 by their healthcare provider. However, the Sofia 2 has been reported to have false negative rate of 20%. Therefore, there exists a need for a specific, sensitive, and rapid diagnostic test for SARS-CoV-2 infection. Further, there exists a need for antibodies against SARS-CoV-2 proteins that could be used in the development of diagnostic testing.

### SUMMARY

In one aspect, the invention provides for a kit for detecting or quantifying a SARS-CoV-2 nucleocapsid phosphoprotein, the kit comprising a first antibody, wherein a variable heavy chain domain of the first antibody comprises the amino acid sequence selected from the group consisting of SEQ ID NO: 1, SEQ ID NO: 3, SEQ ID NO: 5, SEQ ID NO: 7, SEQ ID NO: 11, SEQ ID NO: 13, SEQ ID NO: 15, SEQ ID NO: 17, SEQ ID NO: 19, and SEQ ID NO: 21, and a variable light chain domain of the first antibody comprising the amino acid sequence selected from the group consisting of SEQ ID NO: 2, SEQ ID NO: 4, SEQ ID NO:6, SEQ ID NO: 8, SEQ ID NO: 12, SEQ ID NO: 14, SEQ ID NO: 16, SEQ ID NO: 18, SEQ ID NO: 20, and SEQ ID NO: 22; and a second antibody, wherein a variable heavy chain domain of the second antibody comprises the amino acid sequence selected from the group consisting of SEQ ID NO: 9, and SEQ ID NO: 13, and a variable light chain domain of the second antibody comprises the amino acid sequence selected from the group consisting of SEQ ID NO: 10, and SEQ ID NO: 14.

In some embodiments, the kit includes an ELISA plate having a plurality of compartments. In some embodiments, the plurality of compartments includes a reaction chamber and one or more of the plurality of compartments further includes one of the first antibody or the second antibody described herein. In some embodiments, the kit includes a third antibody, wherein the third antibody is an anti-human antibody having a detectable marker. In some embodiments, the kit includes an enzyme linked to one of the first antibody or the second antibody. In some embodiments, the kit includes a substrate capable of detecting the detectable marker. In some embodiments, the first antibody is a capture antibody and the second antibody is a detection antibody. In some embodiments, the second antibody is a capture antibody and the first antibody is a detection antibody. In some embodiments, the variable heavy chain domain of the first antibody comprises the amino acid sequence of SEQ ID NO: 1 and the variable light chain domain of the first antibody comprises the amino acid sequence of SEQ ID NO: 2. In some embodiments, the variable heavy chain domain of the first antibody comprises the amino acid sequence of SEQ ID NO: 3 and the variable light chain domain of the first antibody comprises the amino acid sequence of SEQ ID NO: 4. In some embodiments, the variable heavy chain domain of the first antibody comprises the amino acid sequence of SEQ ID NO: 5 and the variable light chain domain of the first antibody comprises the amino acid sequence of SEQ ID NO: 6. In some embodiments, the variable heavy chain domain of the first antibody comprises the amino acid sequence of SEQ ID NO: 7 and the variable light chain domain of the first antibody comprises the amino acid sequence of SEQ ID NO: 8. In some embodiments, the variable heavy chain domain of the first antibody comprises the amino acid sequence of SEQ ID NO: 11 and the variable light chain domain of the first antibody comprises the amino acid sequence of SEQ ID NO: 12. In some embodiments, the variable heavy chain domain of the first antibody comprises the amino acid sequence of SEQ ID NO: 13 and the variable light chain domain of the first antibody comprises the amino acid sequence of SEQ ID NO: 14. In some embodiments, the variable heavy chain domain of the first antibody comprises the amino acid sequence of SEQ ID NO: 15 and the variable light chain domain of the first antibody comprises the amino acid sequence of SEQ ID NO: 16. In some embodiments, the variable heavy chain domain of the first antibody comprises the amino acid sequence of SEQ ID NO: 17 and the variable light chain domain of the first antibody comprises the amino acid sequence of SEQ ID NO: 18. In some embodiments, the variable heavy chain domain of the first antibody comprises the amino acid sequence of SEQ ID NO: 19 and the variable light chain domain of the first antibody comprises the amino acid sequence of SEQ ID NO: 20. In some embodiments, the variable heavy chain domain of the first antibody comprises the amino acid sequence of SEQ ID NO: 21 and the variable light chain domain of the first antibody comprises the amino acid sequence of SEQ ID NO: 22. In some embodiments, the variable heavy chain domain of the second antibody comprises the amino acid sequence of SEQ ID NO: 9 and the variable light chain domain of the second antibody comprises the amino acid sequence of SEQ ID NO: 10. In some embodiments, the variable heavy chain domain of the second antibody comprises the amino acid sequence of SEQ ID NO: 13 and the variable light chain domain of the second antibody comprises the amino acid sequence of SEQ ID NO: 14. In some embodiments, each of the first antibody and the second antibody bind to different epitopes of the SARS-CoV-2 nucleocapsid phosphoprotein. In some embodiments, the kit is configured to detect, in a biological sample, the presence of a SARS-CoV-2 nucleocapsid phosphoprotein.

In another aspect, the invention provides for an immunoassay method to detect or quantitate a SARS-CoV-2 nucleocapsid phosphoprotein, the method including coating a first solid surface with a coating antibody selected from one of the first antibody or the second antibody described herein (above), contacting the coated first solid surface with the biological sample to form a complex between a SARS-CoV-2 nucleocapsid phosphoprotein in the sample and the coating antibody, removing unbound biological sample, contacting the coated first solid surface with a detection antibody selected from one of the first antibody and the second antibody to form a complex between the SARS-CoV-2 nucleocapsid phosphoprotein in the sample and the detection antibody, contacting the coated first solid surface with an anti-human antibody having a detectable marker to form a complex between the anti-human antibody and the detection antibody, washing the coated first solid surface, contacting the coated first solid surface with a substrate capable of detecting the detectable marker, and detecting or quantitating the detectable marker of the anti-human antibody.

In some embodiments, the capture antibody is the first antibody described herein (above) and the detection antibody is the second antibody described herein (above). In some embodiments, the capture antibody is the second antibody described herein (above) and the detection antibody is the first antibody described herein (above). In some embodiments, each of the first antibody and the second antibody bind to different epitopes of the SARS-CoV-2 nucleocapsid phosphoprotein. In some embodiments, the detectable marker is horseradish peroxidase. In some embodiments, the substrate is 3,3′,5,5′-Tetramethylbenzidine (TMB). In some embodiments, the biological sample is human serum. In some embodiments, the detecting or quantitating includes measuring optical density at a wavelength of around 450 nm. In some embodiments, the method includes generating a positive test result for a SARS-CoV-2 infection in a subject wherein the measured optical density is above a predetermined value. In some embodiments, the method includes generating a negative test result for a SARS-CoV-2 infection in a subject wherein the measured optical density is below a predetermined value. In some embodiments, the method is capable of detecting the SARS-CoV-2 nucleocapsid phosphoprotein when present in the biological sample at between about ng and 0.02 ng. In some embodiments, the detection or quantitation of the SARS-CoV-2 nucleocapsid phosphoprotein is completed within about 4 hours and within about 1 hour. In some embodiments, the detection or quantitation of the SARS-CoV-2 nucleocapsid phosphoprotein is completed within less than about 1 hour. In some embodiments, the method further includes amplifying a signal using Tyramide Signal Amplification (TSA) before detecting or quantitating the detectable marker of the anti-human antibody.

In another aspect, the invention provides for an immunoassay method to detect or quantitate a SARS-CoV-2 nucleocapsid phosphoprotein, the method including coating a first solid surface with a coating antibody selected from one of the first antibody or the second antibody described herein (above), contacting the coated first solid surface with the biological sample to form a complex between a SARS-CoV-2 nucleocapsid phosphoprotein in the sample and the coating antibody, removing unbound biological sample, contacting the coated first solid surface with a detection antibody selected from one of the first antibody and the second antibody described herein (above) to form a complex between the SARS-CoV-2 nucleocapsid phosphoprotein in the sample and the detection antibody, wherein the detection antibody further having a detectable marker, washing the coated first solid surface, contacting the coated first solid surface with a substrate capable of detecting the detectable marker, and detecting or quantitating the detectable marker of the detection antibody.

In some embodiments, the capture antibody is the first antibody described herein (above) and the detection antibody is the second antibody described herein (above). In some embodiments, the capture antibody is the second antibody described herein (above) and the detection antibody is the first antibody described herein (above). In some embodiments, each of the first antibody and the second antibody bind to different epitopes of the SARS-CoV-2 nucleocapsid phosphoprotein. In some embodiments, the detectable marker is horseradish peroxidase. In some embodiments, the substrate is 3,3′,5,5′-Tetramethylbenzidine (TMB). In some embodiments, the biological sample is human serum. In some embodiments, the detecting or quantitating includes measuring optical density at a wavelength of around 450 nm. In some embodiments, the method includes generating a positive test result for a SARS-CoV-2 infection in a subject, wherein the measured optical density is above a predetermined value. In some embodiments, the method includes generating a negative test result for a SARS-CoV-2 infection in a subject, wherein the measured optical density is below a predetermined value. In some embodiments, the method is capable of detecting the SARS-CoV-2 nucleocapsid phosphoprotein when present in the biological sample at between about 0.001 ng and 0.02 ng. In some embodiments, the detection or quantitation of the SARS-CoV-2 nucleocapsid phosphoprotein is completed within about 4 hours and within about 1 hour. In some embodiments, the detection or quantitation of the SARS-CoV-2 nucleocapsid phosphoprotein is completed within less than about 1 hour. In some embodiments, the method further includes amplifying a signal using Tyramide Signal Amplification (TSA) before detecting or quantitating the detectable marker of the detection antibody.

In another aspect, the invention provides for a purified chimeric monoclonal antibody, or a functional fragment thereof, capable of specifically binding to a SARS-CoV-2 nucleocapsid phosphoprotein, wherein said monoclonal antibody, or functional fragment thereof, comprises any one amino acid sequence selected from the group consisting of heavy chain variable region comprising SEQ ID NO: 17, light chain variable region comprising SEQ ID NO: 18, heavy chain variable region comprising SEQ ID NO: 19, light chain variable region comprising SEQ ID NO: 20, heavy chain variable region comprising SEQ ID NO: 21, and light chain variable region comprising SEQ ID NO: 22.

In some embodiments, the monoclonal antibody comprises heavy chain variable region comprising SEQ ID NO: 17 and light chain variable region comprising SEQ ID NO: 18. In some embodiments, the monoclonal antibody comprises heavy chain variable region comprising SEQ ID NO: 19 and light chain variable region comprising SEQ ID NO: 20. In some embodiments, the monoclonal antibody comprises heavy chain variable region comprising SEQ ID NO: 21 and light chain variable region comprising SEQ ID NO: 22.

### BRIEF DESCRIPTION OF THE DRAWINGS

The following figures depict illustrative embodiments of the invention.

FIGS. 1A-B depict ELISA binding of plasma samples from severe (FIG. 1A) and non-severe (FIG. 1B) COVID-19 patients, specifically of patients' antibody responses to SARS-CoV-2 nucleocapsid phosphoprotein (NP).

FIGS. 2A-B depict a process for identifying and synthesizing binding antibodies against SARS-CoV-2 NP (FIG. 2A) and the sorting results of the isolation of NP-specific memory B cells using flow cytometry (FIG. 2B).

FIG. 3 depicts ELISA monoclonal antibody binding against SARS-CoV-2 NP.

FIG. 4 depicts Western blotting analysis of linear epitope recognition by monoclonal antibody against SARS-CoV-2 NP.

FIGS. 5A-B depict competition ELISA binding of pairs of monoclonal antibodies against SARS-CoV-2 NP (FIG. 5A) and corresponding area under the curve (FIG. 5B).

FIGS. 6A-B depict sandwich ELISA binding for pairs of monoclonal antibodies against SARS-CoV-2 NP that recognize different NP epitopes. FIG. 6A shows ELISA binding for class 1 antibody (9-24) used for capture and class 2 antibodies (9-11, 9-16, 9-17) used as detectors. FIG. 6B shows ELISA binding for class 2 antibodies (9-11, 9-16, 9-17) used for capture and class 1 antibody (9-24) used as a detector (9-24).

FIGS. 7A-B depict SPR sensorgrams of SARS-CoV-2 NP antibodies binding to NP (FIG. 7A) and binding rate constants of the NP antibodies (FIG. 7B).

FIGS. 8A-B depict sandwich ELISA binding for pairs of monoclonal antibodies against SARS-CoV-2 NP that recognize different NP epitopes. FIG. 8A shows ELISA binding for human antibody pairs. FIG. 8B shows ELISA binding for human and chimeric antibody pairs.

FIGS. 9A-C depict sandwich ELISA binding for class 1 antibody (9-24) used for capture and class 2 antibody (chimeric 9-11) used for detection of NP. FIG. 9A shows ELISA binding performed on Vero cell media supernatant (left panel) or lysate (right panel) samples.

FIG. 9B shows ELISA binding performed on saliva samples from healthy donors. FIG. 9C shows ELISA binding using purified SARS-CoV-2 NP.

FIGS. 10A-B depict sandwich ELISA binding for class 1 antibody (9-24) used for capture and class 2 antibody (chimeric 9-11) used for detection of NP. FIG. 10A shows ELISA binding performed on samples spiked with purified SARS-CoV-2 NP with and without saliva. FIG. 10B shows ELISA binding performed on samples spiked with SARS-CoV-2 viral particles (prepared by 1% NP-40 inactivation) with and without saliva.

FIGS. 11A-B depict sandwich ELISA binding for class 1 antibody (9-24) used for capture and class 2 antibody (chimeric 9-11) used for detection of NP. FIG. 11A shows ELISA binding performed according to standard incubation procedures that take about 4 hours to conduct. FIG. 11B shows ELISA binding performed according to shortened incubation procedures that take about 1 hour to conduct.

FIG. 12 depicts mechanisms of Tyramide Signal Amplification (TSA) technology.

FIGS. 13A-B depict sandwich ELISA binding for detection of NP using a standard unamplified procedure (FIG. 13A) and using amplification by TSA technology (FIG. 13B).

FIGS. 14A-C depict the sensitivity and specificity of an antibody pair for detection of SARS-CoV-2 NP. FIG. 14A shows a phylogenetic tree of coronavirus species, FIG. 14B shows a Western blot analysis of various coronavirus NPs, and FIG. 14C shows sandwich ELISA binding for detection of NP for various coronavirus species.

FIGS. 15A-B depict detection of NP from SARS-CoV-2 infected cells (antibody pair: 9-24 and chimeric 9-11). FIG. 15A shows a standard detection curve with dilution and FIG. 15B shows the results of sandwich ELISA binding for detection of SARS-CoV-2 NP across different multiplicities of infection.

### DETAILED DESCRIPTION

In one aspect, the invention provides for an improved antigen sandwich (also known as “capture”) ELISA for the detection of SARS-CoV-2 infection using antibodies against SARS-CoV-2 nucleocapsid phosphoprotein (“NP”) antigens. Antibodies provide strong binding to antigens requiring less amounts of antigens for detection and therefore increased assay sensitivity. An advantage of the disclosed systems and methods is the ability to provide an antigen test that is faster and easier to perform compared to PCR tests yet overcomes the inaccuracy issue regarding false negative readings of other antigen tests.

The present disclosure is directed to the isolation and characterization of sequences of a panel of monoclonal antibodies targeting SARS-CoV-2 NP, the most abundantly expressed immunodominant protein that interacts with RNA. In some embodiments, the present disclosure provides for an improved sandwich ELISA method of detecting SARS-CoV-2 NP antigens by using monoclonal antibodies that target multiple epitopes of the nucleocapsid, thus allowing for the selection of one or more antibody pairs for assay optimization, resulting in high sensitivity and specificity. In some embodiments, the monoclonal against SARS-CoV-2 NP are used for capture assays to detect the presence of SARS-CoV-2 NP antigens in various clinical samples. In some embodiments, the disclosed assays and antibody pairs are used for commercial rapid test kits for COVID-19 antigen detection.

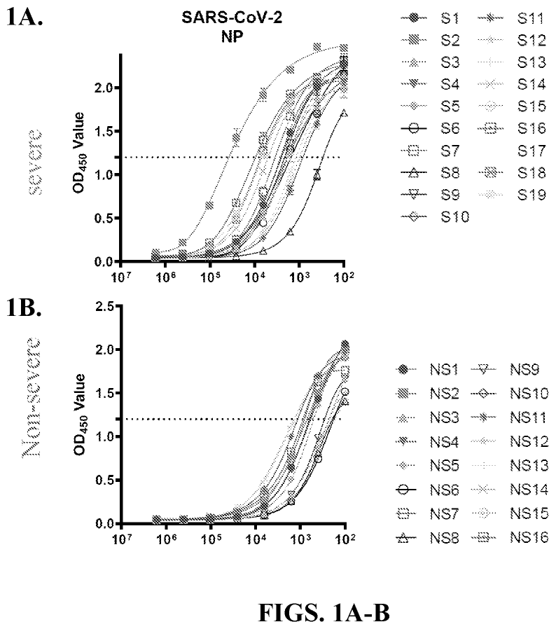

Referring to FIG. 1, two graphs are depicted which show ELISA binding data of plasma samples from COVID-19 patients to SARS-CoV-2 NP. FIG. 1A shows the ELISA binding data for COVID-19 patients having severe disease and FIG. 1B shows the ELISA binding data for COVID-19 patients having non-severe disease. FIG. 1 demonstrates that both severe and non-severe COVID-19 patients develop robust antibody responses to the SARS-CoV-2 NP.

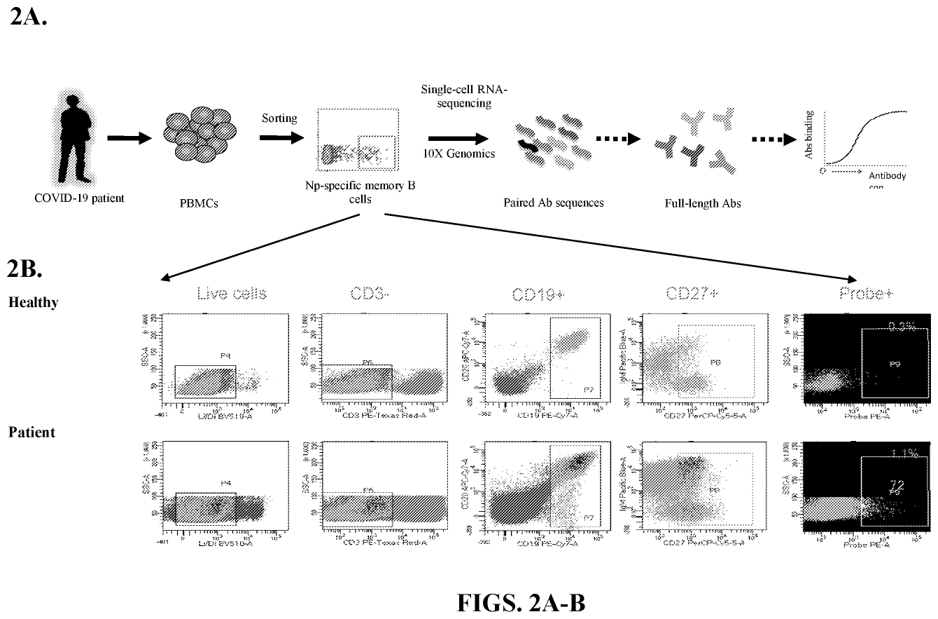

Referring to FIG. 2, a diagram is depicted showing the process for identifying strong binding antibodies against the SARS-CoV-2 NP. Plasma samples from severe and non-severe COVID-19 patients (see FIG. 1) are isolated and evaluated for the ability to bind SARS-CoV-2 NP. From those COVID-19 patients who develop robust antibody responses against NP, plasma samples are subjected to the experimental schema depicted in FIG. 2 in order to identify monoclonal antibodies that could recognize the SARS-CoV-2 NP. Referring to FIG. 2A, peripheral blood mononuclear cells are extracted from COVID-19 patients and antibody-producing cells known as CD19+CD27+ memory B cells are isolated. The focus is on the subset of cells that bind the SARS-CoV-2 NP. Cutting-edge genomics technology (e.g., high throughput sequencing) is used to extract, amplify, and sequence each set of antibody genes allowing for the reconstruction of each monoclonal antibody against SARS-CoV-2 NP. The antibody genes are cloned into expression vectors. In some embodiments, the variable regions of the identified human antibodies are combined with constant regions from mouse antibodies to create chimeric antibodies. The monoclonal antibodies are expressed in vitro and purified for subsequent characterization experiments. FIG. 2B depicts the sorting results of the isolation of NP-specific memory B cells using flow cytometry. Inset numbers indicate the absolute number and the percentage of NP trimer-specific memory B cells isolated from a COVID-19 patient.

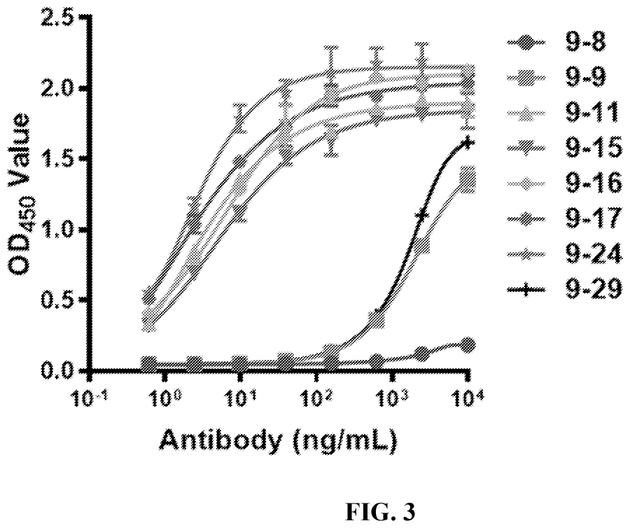

Referring to FIG. 3, monoclonal antibody binding (using ELISA) is depicted for a panel of monoclonal antibodies against SARS-CoV-2 NP which were synthesized using the methods described herein. FIG. 3 shows robust binding of the synthesized monoclonal antibodies to SARS-CoV-2 NP.

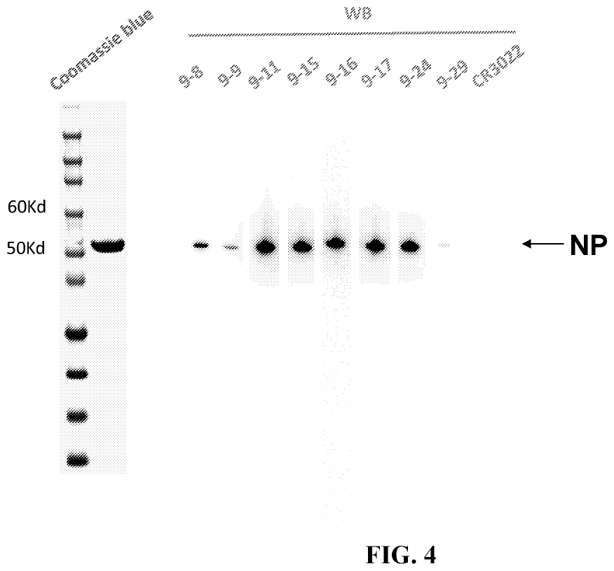

Referring to FIG. 4, Western blotting analyses were performed to test whether synthesized SARS-CoV-2 NP-specific antibodies recognize linear epitopes on the SARS-CoV-2 NP. Synthesized human antibodies are indicated as 9-8, 9-9, 9-11, 9-15, 9-16, 9-17, 9-24 and 9-29. Before blotting with each antibody, SARS-CoV-2 NP was denatured and linearized by treating with dithiothreitol (DTT) and heat, and then running NuPAGE®. CR3022 is a SARS-CoV-2 spike trimer-specific antibody binding the receptor binding domain (RBD) region. FIG. 4 shows that the synthesized NP antibodies recognize the linear epitope of NP and are specific as they do not recognize the spike protein (CR3022). In some embodiments, the synthesized antibodies are able to detect the SARS-CoV-2 NP from the physically and chemically inactivated SARS-CoV-2 virus.

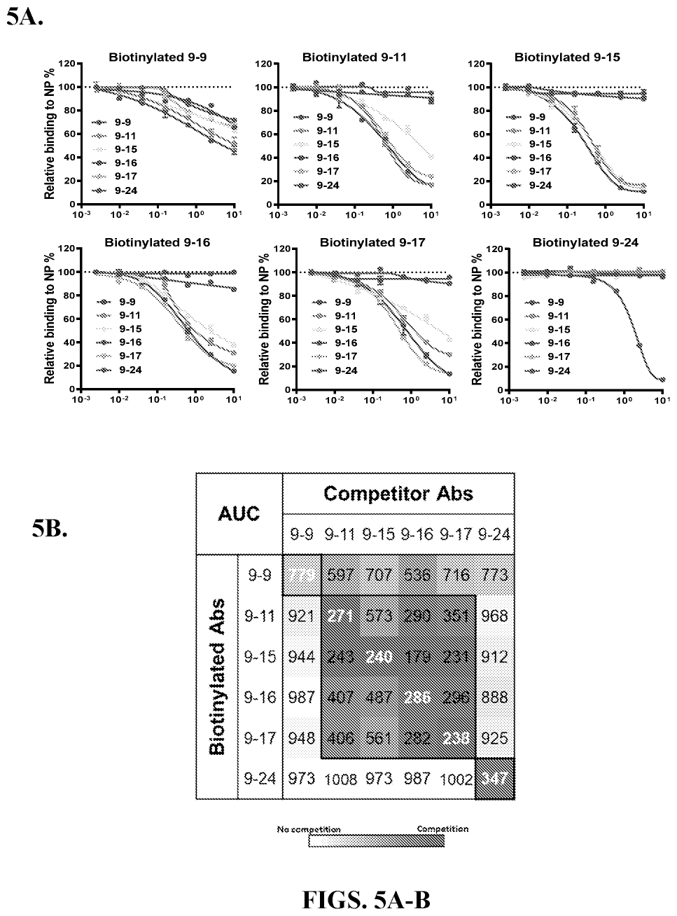

Referring to FIG. 5, epitope mapping is depicted for SARS-CoV-2 NP-specific human antibodies by competition ELISA. In order to determine the epitope of the binding antibodies on SARS-CoV-2 NP, competition ELISAs were performed. FIG. 5A depicts competition ELISA curves for six synthesized antibodies: 9-9, 9-11, 9-16, 9-15, 9-17 and 9-24. For each graph, competition is shown between the biotinylated antibody (labeled at the top of each graph) and the other five antibodies. FIG. 5B shows the area under the curve (AUC) from FIG. 5A. Based on the competition ELISA data, the NP-specific antibodies are categorized into three classes: class 1 only contains antibody 9-24, class 2 contains antibodies 9-11, 9-15, 9-16 and 9-17, and class 3 only contains antibody 9-9, There is no competition between the three classes of antibodies, meaning that antibodies in the three classes bind noncompetitively to different epitopes on the SARS-CoV-2 NP. This allows for selection of pairs of antibodies across the three classes for development of a sandwich ELISA with high sensitivity as described herein.

In some embodiments, the monoclonal antibodies are capable of specifically binding a N-terminal domain of the SARS-CoV-2 virus. In some embodiments, the monoclonal antibodies are incapable of specifically binding a N-terminal domain of the SARS-CoV-2 virus. In some embodiments, antibodies capable of specifically binding a N-terminal domain and antibodies incapable of specifically binding a N-terminal domain do not compete for epitope binding.

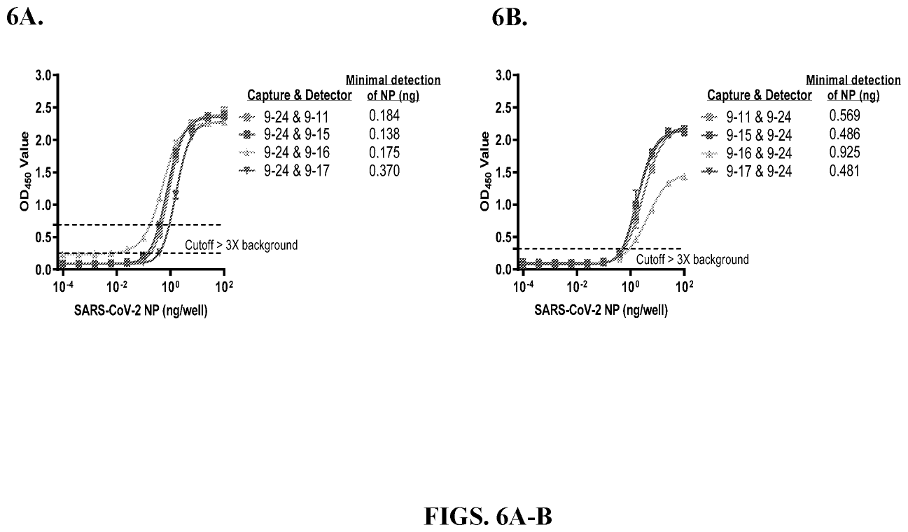

Referring to FIG. 6, ELISA binding is shown for sandwich ELISAs of human antibodies of the current disclosure. The graphs show the limit of NP that can be detected by some embodiments of the sandwich ELISA using the different combinations of SARS-CoV-2 NP-specific antibodies targeting different epitopes. FIG. 6A shows ELISA binding for class 1 antibody 9-24 which was coated on the ELISA plate and used to capture NP, and biotinylated class 2 antibodies that were applied as detectors (9-11, 9-15, 9-16, and 9-17). FIG. 6B shows the converse in which class 2 antibodies (9-11, 9-15, 9-16, and 9-17) were used as capture antibodies and class 1 antibody 9-24 was used as the detector. The sandwich ELISA can detect as low as less than 0.138 ng of purified NP.

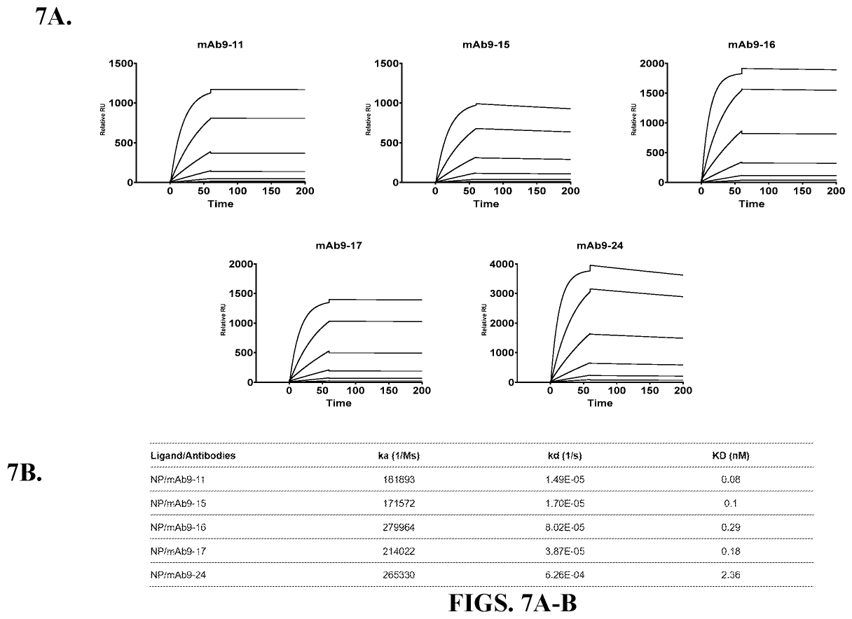

Referring to FIG. 7, binding affinities of monoclonal antibodies 9-11, 9-15, 9-16, 9-17, and 9-24 to SARS-CoV-2 NP are shown. FIG. 7A shows Surface Plasmon Resonance (SPR) sensorgrams of SARS-CoV-2 NP antibodies binding to NP. The NP was immobilized onto CM5 sensor chip at a concentration of 20 μg/ml and NP antibodies were injected at concentrations of 300 nM, 100 nM, 33.3 nM, 11.1 nM, 3.3 nM, and 1.1 nM. FIG. 7B shows the binding rate constants and affinities of NP antibodies (ka=association rate constant; kd=dissociation rate constant; KD=equilibrium constant). Binding affinities were strong for all five antibodies.

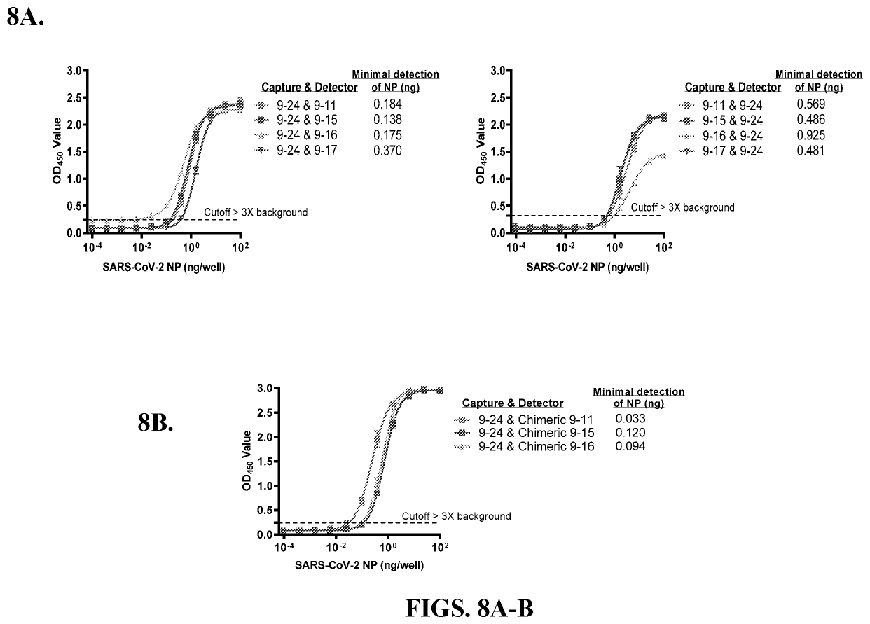

Referring to FIG. 8, ELISA binding is shown for sandwich ELISAs of human and chimeric antibodies of the current disclosure. FIG. 8A shows sandwich ELISA binding for the detection of NP using human antibody 9-24 (left panel; class 1) or human antibodies 9-11, 9-15, 9-16 and 9-17 (right panel; class 2) as the capture antibodies paired with biotinylated human antibodies 9-11, 9-15, 9-16 and 9-17 (left panel; class 2) or biotinylated human antibody 9-24 (right panel; class 1) as the detection antibodies. The minimal detection of NP was defined as the value corresponding to 3-fold higher optical density (OD) value than background. FIG. 8B shows sandwich ELISA binding for the detection of NP where the capture antibody is human antibody 9-24 (class 1) and the detector antibodies were chimeric antibodies (chimeric 9-11, chimeric 9-15, and chimeric 9-16; class 2) bearing human variable regions and mouse constant regions. The minimal detection of NP is calculated as the OD value corresponding to 3-fold higher than background. FIG. 8B demonstrates that ELISAs using chimeric detection antibodies provide for increased NP detection sensitivity.

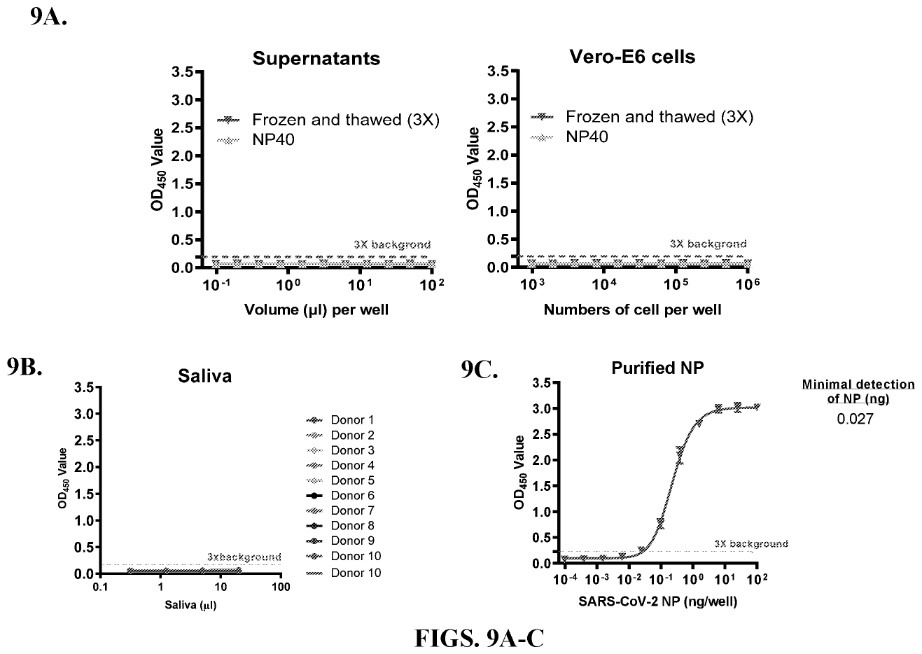

Referring to FIG. 9, ELISA binding is shown for detection of NP using human antibody 9-24 (class 1) as the capture antibody and chimeric antibody 9-11 (class 2) as the detection antibody. FIG. 9A shows ELISA binding performed on Vero cell growth media supernatant (left panel) or lysate of cells (right panel) treated with 1% NP-40 or used after freeze/thaw without treatment (i.e., no NP-40). FIG. 9B shows ELISA binding for detection of NP performed on saliva samples from ten healthy donors treated with 1% NP-40. FIG. 9C shows ELISA binding for detection of NP performed on purified SARS-CoV-2 NP as a positive assay control. In all cases, the minimal detection of NP is calculated as the OD value corresponding to 3-fold higher than background. FIG. 9 demonstrates that NP detection using sandwich ELISAs of the current disclosure is specific since NP was not detected using Vero cell media (FIG. 9A) or saliva from healthy patients (FIG. 9B), and was only detected in samples containing SARS-CoV-2 NP (FIG. 9C).

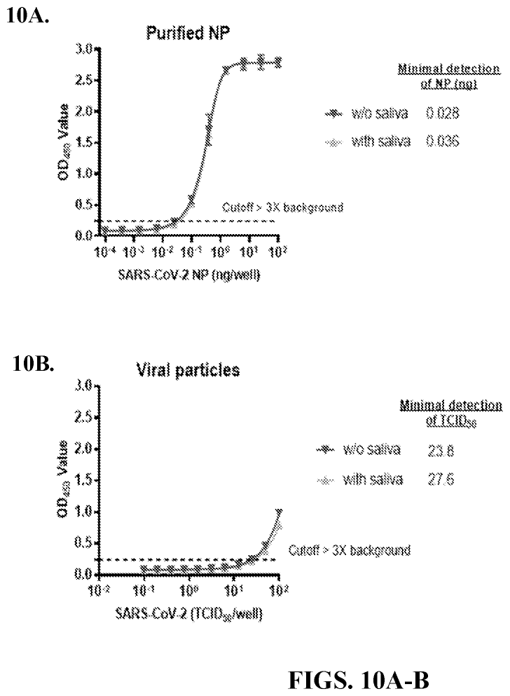

Referring to FIG. 10, sandwich ELISAs are shown as performed on saliva samples using an antibody pair consisting of human antibody 9-24 (class 1) as the capture antibody and chimeric antibody 9-11 (class 2) as the detection antibody. SARS-CoV-2 NP (FIG. 10A) or viral particles (FIG. 10B) were spiked into the saliva (“with saliva”). Purified NP or viral particles (without saliva) were used as controls (“w/o saliva”). Viral particles were prepared by 1% NP-40 inactivation of the SARS-CoV-2 virus. FIG. 10 demonstrates that the presence of saliva does not interference with the ability of the sandwich ELISAs to detect SARS-CoV-2 NP.

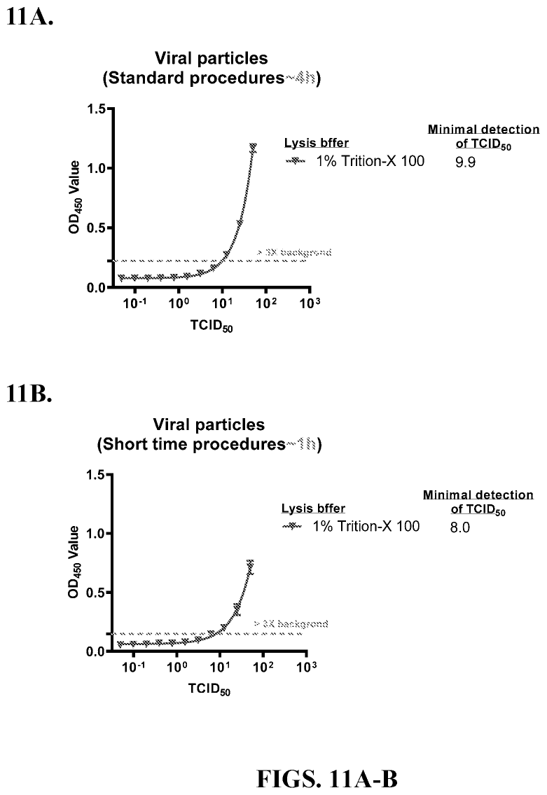

Referring to FIG. 11, viral particles were quantified by two sandwich ELISAs using an antibody pair consisting of human antibody 9-24 (class 1) as the capture antibody and chimeric antibody 9-11 (class 2) as the detection antibody. FIG. 11A shows ELISA binding for detection of NP using a standard incubation procedure (which takes approximately 4 hours in total to perform) including antigen incubation for 1 hour, detection antibody incubation for 1 hour, second antibody (against the detection antibody) incubation for 1 hour, and wash steps performed for 1 hour. FIG. 11B shows ELISA binding for detection of NP using a shorter incubation procedure (which takes approximately 1 hour in total to perform) including combination of antigen and detection antibody incubation for 25 minutes, second antibody incubation for 25 minutes, and wash steps for 10 minutes. The minimal detection of viral particles was compared between the two procedures (FIGS. 11A and 11B) and is shown to be similar.

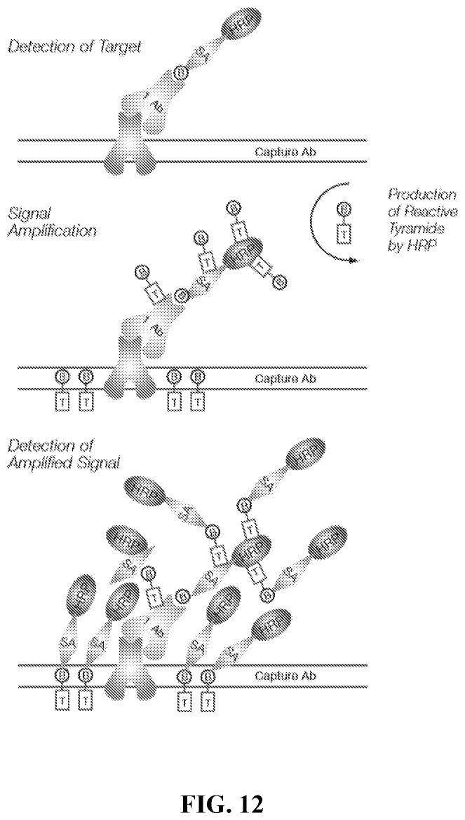

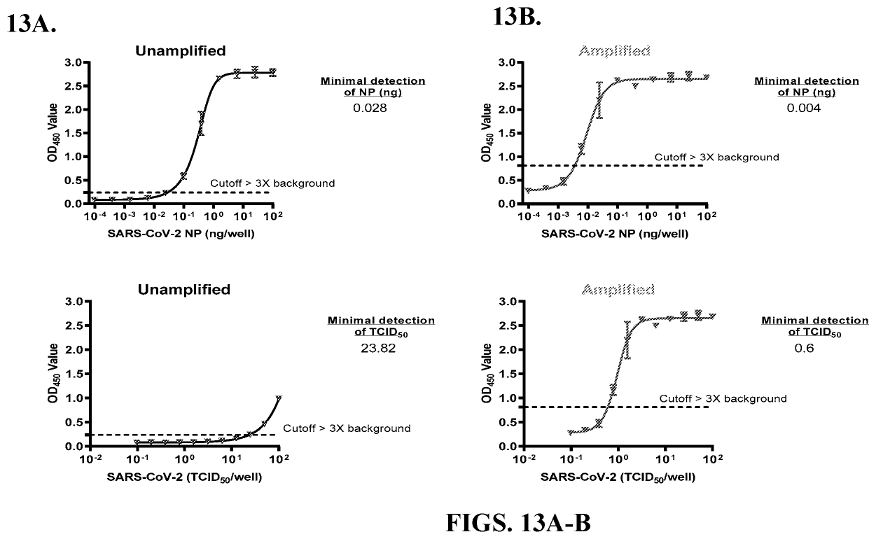

Referring to FIGS. 12 and 13, optimization of the sandwich ELISAs by signal amplification was tested using Tyramide Signal Amplification (TSA) technology. FIG. 12 is a schematic showing the mechanisms of TSA technology. FIG. 13 shows ELISA binding for the detection of NP and resulting minimal sensitivity obtained using read-out from a conventional strategy (“unamplified,” FIG. 13A) or using TSA which adds gain in sensitivity (“amplified,” FIG. 13B). The top panels of FIGS. 13A and 13B show OD values for various levels of SARS-CoV-2 NP measured in ng/well concentration, while the bottom panels show OD values for various levels of SARS-CoV-2 NP measured in terms of fifty-percent-tissue-culture-infective-dose (TCID₅₀). The minimal detection of NP is calculated as the OD value corresponding to 3-fold higher than background. While the background is enhanced, the enhancement in sensitivity using TSA (FIG. 13B) of the detection is between 7-fold (purified NP) to 39-fold (viral particle) for the samples.

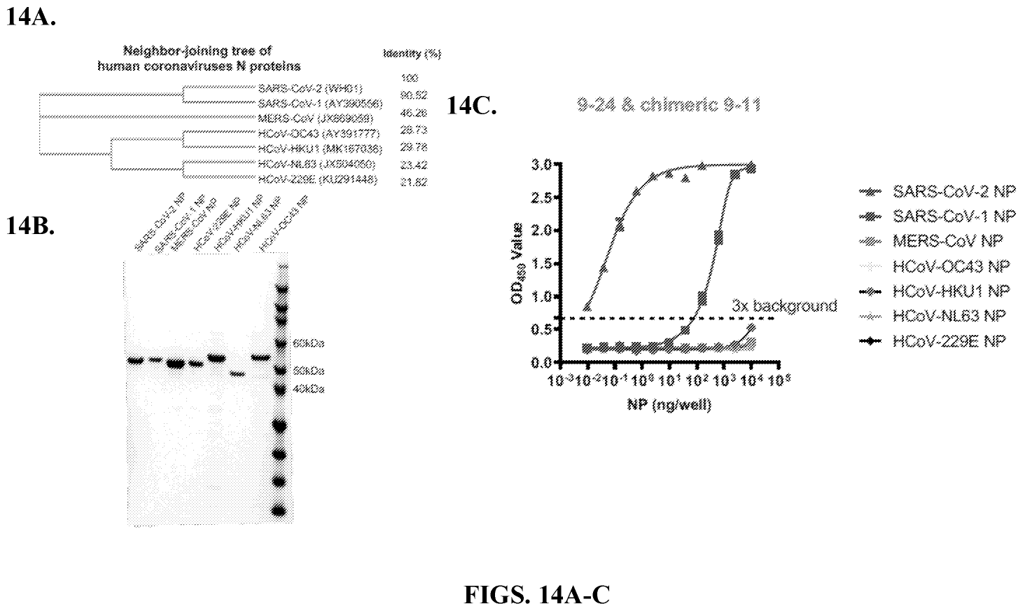

Referring to FIGS. 14A-C, the sensitivity and specificity of the antibody pair for detection of SARS-CoV-2 NP is shown. FIG. 14A shows a phylogenetic tree of various coronavirus species relative to SARS-CoV-2 (WH01), based on the alignment of spike protein sequences. FIG. 14B shows phylogenetic analysis that was conducted by the neighbor-joining method using MEGA 5.0. SDS-PAGE analysis of different Coronavirus NPs. The protein molecular weight marker (kDa) is indicated on the right. FIG. 14C shows a sandwich ELISA employing a pair of monoclonal antibodies (9-24 and chimeric 9-11) to test specificity for different Coronavirus NPs. As shown in FIG. 14C, this antibody pair shows high specificity and sensitivity for SARS-CoV-2 NP, but not for the NPs of the other coronavirus species. While SARS-CoV-1 could also be detected, it was detected at higher concentrations of NP relative to the detection of SARS-CoV-2, thus showing that the ELISA is more sensitive for SARS-CoV-2 compared to any other tested coronavirus species.

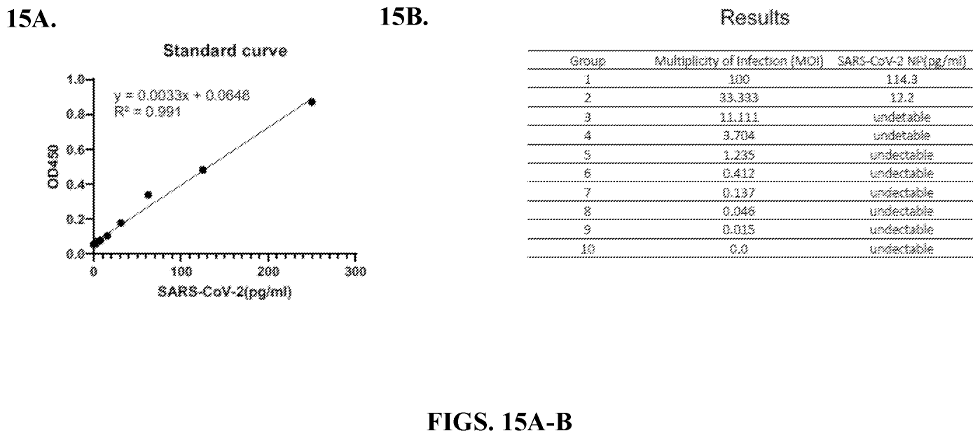

Referring to FIGS. 15A-B, the detection of NP from SARS-CoV-2 infected cells (antibody pair: 9-24 & chimeric 9-11) is shown. 0.5M cells were infected with SARS-CoV-2 at different multiplicity of infection (MOI). Cells were harvested at 1.5 hours after infection, washed in PBS, then lysed in PBS with 1% TritonX-100. After lysis, soluble NP was measured by a sandwich ELISA as described herein. Referring to FIG. 15A, the standard was diluted from 250 pg/ml to 0 pg/ml. Referring to FIG. 15B, the NPs of the infected cells were measured by the sandwich assay. SARS-CoV-2 NP was detectable for MOIs of 100 and 33.333 (Groups 1 and 2), but not for the other, lower MOIs.

### Sequences of Human Monoclonal Antibodies to SARS-CoV-2 NP

Underlined and italicized amino acids represent the respective complementarity determining regions (CDRs).

In some embodiments, the amino acid sequence of the variable heavy chain domain of an anti-SARS-CoV-2 NP antibody comprises SEQ ID NO: 1. QVQLVQSGAEVKKPGASVKVSCKASGYTFTSYAISWVRQAPGQGLEWMGWISAYTGN TlVYAQKLQGRVTMTTDTSTSTAYMELRSLRSDDT AVYYCARNGWDYDTSGTHDYWG QGTLVTVSS (SEQ ID NO: 1). The corresponding variable light chain domain of the anti-SARS-CoV-2 NP antibody comprises SEQ ID NO: 2. EIVLTQSPGTLSLSPGERATLSCRASQSVSSSYLAWYQQKPGQAPRLLIYGASSRATGIPD RFSGSGSGTDFTLTISRLEPEDFAVYYCQQYGSSPRTFGQGTKVEIK (SEQ ID NO: 2). The antibody comprising SEQ ID NO: 1 and SEQ ID NO: 2 is represented by antibody “9-11” in embodiments described and depicted in this disclosure.

In some embodiments, the variable heavy or light chain domains of the anti-SARS-CoV-2 NP antibody comprise an amino acid sequence 80, 81, 82, 83, 84, 85, 86, 87, 88, 89, 90, 91, 92, 93, 94, 95, 96, 97, 98 or 99% identical to SEQ ID NO: 1 or SEQ ID NO: 2.

In some embodiments, the amino acid sequence of the variable heavy chain domain of an anti-SARS-CoV-2 NP antibody comprises SEQ ID NO: 3. QVQLVQSGAEVKKPGSSVKVSCKASGGTFSSYAISWVRQAPGQGLEWMGGIIPIFGTA NYAQKFQGRVTITAEESTSTAYMELSSLRSEDTAVYYCARDGWAAAGPDTSLLGTFDI WGQGTMVTVSS (SEQ ID NO: 3). The corresponding variable light chain domain of the anti-SARS-CoV-2 NP antibody comprises SEQ ID NO: 4. QSALTQPPSVSGSPGQSVTISCTGTSSDVGSYNRVSWYQQPPGTAPKLIIYEVSNRPSGVP DRFSGSKSGNTASLTISGLQAEDEADYYCSSYTSSSTYVVFGGGTKLTVL (SEQ ID NO: 4). The antibody comprising SEQ ID NO: 3 and SEQ ID NO: 4 is represented by antibody “9-15” in embodiments described in this disclosure.

In some embodiments, the variable heavy or light chain domains of the anti-SARS-CoV-2 NP antibody comprise an amino acid sequence 80, 81, 82, 83, 84, 85, 86, 87, 88, 89, 90, 91, 92, 93, 94, 95, 96, 97, 98 or 99% identical to SEQ ID NO: 3 or SEQ ID NO: 4.

In some embodiments, the amino acid sequence of the variable heavy chain domain of an anti-SARS-CoV-2 NP antibody comprises SEQ ID NO: 5. QVQLVQSGAEVKKPGSSVKVSCKASGGTFSSYAISWVRQAPGQGLEWMGGIIPIFGTA NYAQKFQGRVTITADESTSTAYMELSSLRSEDTAVYYCARTSWGSGSYYKTYYYNGMD VWGQGTTGTVSS (SEQ ID NO: 5). The corresponding variable light chain domain of the anti-SARS-CoV-2 NP antibody comprises SEQ ID NO: 6. QSVLTQPPSASGTPGQRVTISCSGSSSNIGSNTVNVWYQQLPGTAPKLLIYTNNQRPSGVP DRFSGSKSGTSASLAISGLQSEDEADYYCAAWDDSLNGRYVVFGGGTKLTVL (SEQ ID NO: 6). The antibody comprising SEQ ID NO: 5 and SEQ ID NO: 6 is represented by antibody “9-16” in embodiments described and depicted in this disclosure.

In some embodiments, the variable heavy or light chain domains of the anti-SARS-CoV-2 NP antibody comprise an amino acid sequence 80, 81, 82, 83, 84, 85, 86, 87, 88, 89, 90, 91, 92, 93, 94, 95, 96, 97, 98 or 99% identical to SEQ ID NO: 5 or SEQ ID NO: 6.

In some embodiments, the amino acid sequence of the variable heavy chain domain of an anti-SARS-CoV-2 NP antibody comprises SEQ ID NO: 7. QVQLVQSGAEVKKPGSSVKVSCKASGGTFSNYVISSWVRQAPGQGLEWMGGIIPIFGT AKYAQKFQGRVAITADESTSTAYMEVSSLRSEDTAVYYCARAGYCSGGSCRRPSDYYG MDVWGQGTTVTVSS (SEQ ID NO: 7). The corresponding variable light chain domain of the anti-SARS-CoV-2 NP antibody comprises SEQ ID NO: 8. DIVMTQSPLSLPVTPGEPASISCRSSQSLLHSNGYNYLDWYLQKPGQSPQLLIYLGSNRA SGVPDRFSGSGSGTDFTLKISRVEAEDVGVYYCMQALQTPRTFGQGTKLEIK (SEQ ID NO: 8). The antibody comprising SEQ ID NO: 7 and SEQ ID NO: 8 is represented by antibody “9-17” in embodiments described and depicted in this disclosure.

In some embodiments, the variable heavy or light chain domains of the anti-SARS-CoV-2 NP antibody comprise an amino acid sequence 80, 81, 82, 83, 84, 85, 86, 87, 88, 89, 90, 91, 92, 93, 94, 95, 96, 97, 98 or 99% identical to SEQ ID NO: 7 or SEQ ID NO: 8.

In some embodiments, the amino acid sequence of the variable heavy chain domain of an anti-SARS-CoV-2 NP antibody comprises SEQ ID NO: 9. EVQLVESGGGLVQPGGSLRLSCAASGFTFSDSAVHWVRQASGKGLEWVGRIRNRNNN YATAYAASVKGRFTISRDDSENMAYLQMNGLKTEDTAIYYCTDLLAYWGQGTLLTVSS (SEQ ID NO: 9). The corresponding variable light chain domain of the anti-SARS-CoV-2 NP antibody comprises SEQ ID NO: 10. SYELTQPPSVSVSPGQTARITCSADALPKQYAYWYQQKAGQAPVLVIYKDNERPSGIPE RFSGSSSGTTVTLTISGVQAEDEADYYCQSADSSGGYRVFGGGTKLTVL (SEQ ID NO: The antibody comprising SEQ ID NO: 9 and SEQ ID NO: 10 is represented by antibody “9-24” in embodiments described and depicted in this disclosure.

In some embodiments, the variable heavy or light chain domains of the anti-SARS-CoV-2 NP antibody comprise an amino acid sequence 80, 81, 82, 83, 84, 85, 86, 87, 88, 89, 90, 91, 92, 93, 94, 95, 96, 97, 98 or 99% identical to SEQ ID NO: 9 or SEQ ID NO:

In some embodiments, the amino acid sequence of the variable heavy chain domain of an anti-SARS-CoV-2 NP antibody comprises SEQ ID NO: 11. EVQLVESGGGLVQPGGSLRLSCAASGFTFSTYWMSWVRQAPGKGLEWVANIKQDGS EKYYVDSVKGRFTISRDNAKNSLYLQMNSLRAEDT AVYYCARYSLDYYDTSGSFDYWG QGTLVAVSS (SEQ ID NO: 11). The corresponding variable light chain domain of the anti-SARS-CoV-2 NP antibody comprises SEQ ID NO: 12. SYELTQPPSVSVSPGQTARITCSGDALPKKYAYWYQQKSGQAPVLVIYEDSKRPSGIPE RFSGSSSGTMATLTISGAQVEDEADYYCYSTDSSGNHRGVFGGGTQLTVL (SEQ ID NO: 12). The antibody comprising SEQ ID NO: 11 and SEQ ID NO: 12 is represented by antibody “9-8” in embodiments described and depicted in this disclosure.

In some embodiments, the variable heavy or light chain domains of the anti-SARS-CoV-2 NP antibody comprise an amino acid sequence 80, 81, 82, 83, 84, 85, 86, 87, 88, 89, 90, 91, 92, 93, 94, 95, 96, 97, 98 or 99% identical to SEQ ID NO: 11 or SEQ ID NO: 12.

In some embodiments, the amino acid sequence of the variable heavy chain domain of an anti-SARS-CoV-2 NP antibody comprises SEQ ID NO: 13. EVQLVESGGGLVQPGGSLRLSCAASGFIFSNYWMSWVRQAPGKGLEWVANTKQDDS EKYYVDAVKGRFTISRDNAKNSLYLQMNSLRADDTAVYYCAREVRIAVTGTSRDEDYS YNGMDVWGQGTTVTVSS (SEQ ID NO: 13). The corresponding variable light chain domain of the anti-SARS-CoV-2 NP antibody comprises SEQ ID NO: 14. SYELTQPPSVSVSPGQTARITCSADALAKQYAYWYQQKPGQAPVLVIFKDSERPSGIPER FSGSSSGTTVTLTISRVQAEDEADYYCQSADSSGYYWAFGGGTKLTVL (SEQ ID NO: 14). The antibody comprising SEQ ID NO: 13 and SEQ ID NO: 14 is represented by antibody “9-9” in embodiments described and depicted in this disclosure.

In some embodiments, the variable heavy or light chain domains of the anti-SARS-CoV-2 NP antibody comprise an amino acid sequence 80, 81, 82, 83, 84, 85, 86, 87, 88, 89, 90, 91, 92, 93, 94, 95, 96, 97, 98 or 99% identical to SEQ ID NO: 13 or SEQ ID NO: 14.

In some embodiments, the amino acid sequence of the variable heavy chain domain of an anti-SARS-CoV-2 NP antibody comprises SEQ ID NO: 15. EVQLVQSGAEVKKSGESLKISCKGSGYSFINYWIGWVRQMPGKGLEWMGIIYPGDSD TRHSPSFQGQVTISADKSLRTAYLQWSSLKASDTAIYYCARGADGYSSYFDYWGQGTL VTVSS (SEQ ID NO: 15). The corresponding variable light chain domain of the anti-SARS-CoV-2 NP antibody comprises SEQ ID NO: 16. NFMLTQPHSVSESPGKTVIISCTRSSGSIASDYVQWYQQRPGSVPTTVIYEDNERPSGVP DRFSGSIDSSSNSASLTISGLKTEDEADYYCQSYDSSVQVFGGGTKLTVL (SEQ ID NO: 16). The antibody comprising SEQ ID NO: 15 and SEQ ID NO: 16 is represented by antibody “9-29” in embodiments described and depicted in this disclosure.

In some embodiments, the variable heavy or light chain domains of the anti-SARS-CoV-2 NP antibody comprise an amino acid sequence 80, 81, 82, 83, 84, 85, 86, 87, 88, 89, 90, 91, 92, 93, 94, 95, 96, 97, 98 or 99% identical to SEQ ID NO: 15 or SEQ ID NO: 16.

### Sequences of Chimeric Monoclonal Antibodies to SARS-CoV-2 NP

Underlined and italicized amino acids represent the respective complementarity determining regions (CDRs).

In some embodiments, the amino acid sequence of the variable heavy chain domain of an anti-SARS-CoV-2 NP chimeric antibody comprises SEQ ID NO: 17. QVQLVQSGAEVKKPGASVKVSCKASGYTFTSYAISWVRQAPGQGLEWMGWISAYTGN TNYAQKLQGRVTMTTDTSTSTAYMELRSLRSDDTAVYYCARNGWDYDTSGTHDYWG QGTLVTVSSASTTAPSVYPLAPVCGDTTGSSVTLGCLVKGYFPEPVTLTWNSGSLSSG VHTFPAVLQSDLYTLSSSVTVTSSTWPSQSITCNVAHPASSTKVDKKIEPRGPTIKPCP PCKCPAPNLLGGPSVFIFPPKIKDVLMISLSPIVTCVVVDVSEDDPDVQISWFVNNVEV HTAQTQTHREDYNSTLRVVSALPIQHQDWMSGKEFKCKVNNKDLPAPIERTISKPKG SVRAPQVYVLPPPEEEMTKKQVTLTCMVTDFMPEDIYVEWTNNGKTELNYKNTEPV LDSDGSYFMYSKLRVEKKNWVERNSYSCSVVHEGLHNHHTTKSFSRTPGK (SEQ ID NO: 17). The corresponding variable light chain domain of the anti-SARS-CoV-2 NP antibody comprises SEQ ID NO: 18. EIVLTQSPGTLSLSPGERATLSCRASQSVSSSYLAWYQQKPGQAPRLLIYGASSRATGIPD RFSGSGSGTDFTLTISRLEPEDFAVYYCQQYGSSPRTFGQGTKVEIKRTDAAPTVSIFPP SSEQLTSGGASVVCFLNNFYPKDINVKWKIDGSERQNGVLNSWTDQDSKDSTYSMS STLTLTKDEYERHNSYTCEATHKTSTSPIVKSFNRNEC (SEQ ID NO: 18). The antibody comprising SEQ ID NO: 17 and SEQ ID NO: 18 is represented by antibody “chimeric 9-11” in embodiments described and depicted in this disclosure.

In some embodiments, the variable heavy or light chain domains of the anti-SARS-CoV-2 NP antibody comprise an amino acid sequence 80, 81, 82, 83, 84, 85, 86, 87, 88, 89, 90, 91, 92, 93, 94, 95, 96, 97, 98 or 99% identical to SEQ ID NO: 17 or SEQ ID NO: 18.

In some embodiments, the amino acid sequence of the variable heavy chain domain of an anti-SARS-CoV-2 NP chimeric antibody comprises SEQ ID NO: 19. QVQLVQSGAEVKKPGSSVKVSCKASGGTESSYAISWVRQAPGQGLEWMGGIIPIEGTA NYAQKFQGRVTITAEESTSTAYMELSSLRSEDTAVYYCARDGWAAAGPDTSLLGTFDI WGQGTMVTVSSASTTAPSVYPLAPVCGDTTGSSVTLGCLVKGYFPEPVTLTWNSGS LSSGVHTFPAVLQSDLYTLSSSVTVTSSTWPSQSITCNVAHPASSTKVDKKIEPRGPTI KPCPPCKCPAPNLLGGPSVFIFPPKIKDVLMISLSPIVTCVVVDVSEDDPDVQISWFVN NVEVHTAQTQTHREDYNSTLRVVSALPIQHQDWMSGKEFKCKVNNKDLPAPIERTIS KPKGSVRAPQVYVLPPPEEEMTKKQVTLTCMVTDFMPEDIYVEWTNNGKTELNYK NTEPVLDSDGSYFMYSKLRVEKKNWVERNSYSCSVVHEGLHNHHTTKSFSRTPGK (SEQ ID NO: 19). The corresponding variable light chain domain of the anti-SARS-CoV-2 NP antibody comprises SEQ ID NO: 20. QSALTQPPSVSGSPGQSVTISCTGTSSDVGSYNRVSWYQQPPGTAPKLIIYEVSNRPSGVP DRFSGSKSGNTASLTISGLQAEDEADYYCSSYTSSSTYVVFGGGTKLTVLGQPKAAPSV TLFPPSSEELKENKATLVCLISNFSPSGVTVAWKANGTPITQGVDTSNPTKEGNKFMA SSFLHLTSDQWRSHNSFTCQVTHEGDTVEKSLSPAECL (SEQ ID NO: 20). The antibody comprising SEQ ID NO: 19 and SEQ ID NO: 20 is represented by antibody “chimeric 9-15” in embodiments described and depicted in this disclosure.

In some embodiments, the variable heavy or light chain domains of the anti-SARS-CoV-2 NP antibody comprise an amino acid sequence 80, 81, 82, 83, 84, 85, 86, 87, 88, 89, 90, 91, 92, 93, 94, 95, 96, 97, 98 or 99% identical to SEQ ID NO: 19 or SEQ ID NO: 20.

In some embodiments, the amino acid sequence of the variable heavy chain domain of an anti-SARS-CoV-2 NP chimeric antibody comprises SEQ ID NO: 21. QVQLVQSGAEVKKPGSSVKVSCKASGGTFSSYAISWVRQAPGQGLEWMGGIINFGTA NYAQKFQGRVTITADESTSTAYMELSSLRSEDTAVYYCARTSWGSGSYYKTYYYNGMD VWGQGTTGTVSSASTTAPSVYPLAPVCGDTTGSSVTLGCLVKGYFPEPVTLTWNSGS LSSGVHTFPAVLQSDLYTLSSSVTVTSSTWPSQSITCNVAHPASSTKVDKKIEPRGPTI KPCPPCKCPAPNLLGGPSVFIFPPKIKDVLMISLSPIVTCVVVDVSEDDPDVQISWFVN NVEVHTAQTQTHREDYNSTLRVVSALPIQHQDWMSGKEFKCKVNNKDLPAPIERTIS KPKGSVRAPQVYVLPPPEEEMTKKQVTLTCMVTDFMPEDIYVEWTNNGKTELNYK NTEPVLDSDGSYFMYSKLRVEKKNWVERNSYSCSVVHEGLHNHHTTKSFSRTPGK (SEQ ID NO: 21). The corresponding variable light chain domain of the anti-SARS-CoV-2 NP antibody comprises SEQ ID NO: 22. QSVLTQPPSASGTPGQRVTISCSGSSSNIGSNTVNWYQQLPGTAPKLLIYTNNQRPSGVP DRFSGSKSGTSASLATSGLQSEDEADYYCAAWDDSLNGRYVVFGGGTKLTVLGQPKA APSVTLFPPSSEELKENKATLVCLISNFSPSGVTVAWKANGTPITQGVDTSNPTKEGN KFMASSFLHLTSDQWRSHNSFTCQVTHEGDTVEKSLSPAECL (SEQ ID NO: 22). The antibody comprising SEQ ID NO: 21 and SEQ ID NO: 22 is represented by antibody “chimeric 9-16” in embodiments described and depicted in this disclosure.

In some embodiments, the variable heavy or light chain domains of the anti-SARS-CoV-2 NP antibody comprise an amino acid sequence 80, 81, 82, 83, 84, 85, 86, 87, 88, 89, 90, 91, 92, 93, 94, 95, 96, 97, 98 or 99% identical to SEQ ID NO: 21 or SEQ ID NO: 22.

In some embodiments, a sandwich ELISA method is used to detect the presence of SARS-CoV-2 NP in a biological sample. The ELISA plate may be coated with the class 1 human antibody 9-24 to capture the SARS-CoV-2 NP and one of the antibodies from class 2 (human antibodies 9-11, 9-15, 9-16, or 9-17; or chimeric antibodies 9-11, 9-15, or 9-16) is used as a detection antibody. In some embodiments, the antibody classes are reversed such that one of the antibodies from class 2 (human antibodies 9-11, 9-15, 9-16, or 9-17; or chimeric antibodies 9-11, 9-15, or 9-16) is coated on the ELISA plate to capture the SARS-CoV-2 NP, and the class 1 human antibody 9-24 is used as a detection antibody. In some embodiments, the detection antibody (either from class 1 or 2) is enzyme-linked (e.g., horseradish peroxidase) such that a substrate (e.g., 3,3′,5,5′-Tetramethylbenzidine) can be applied to detect the presence of SARS-CoV-2 NP.

In some embodiments, the detection antibody is not enzyme-linked and an anti-human antibody that is enzyme-linked (e.g., horseradish peroxidase) is used to bind to the detection antibody (from class 1 or 2) such that a substrate (e.g., 3,3′,5,5′-Tetramethylbenzidine) can be applied to detect the presence of SARS-CoV-2 NP. In such an embodiment, a sandwich ELISA would require three antibodies: a capture antibody (selected from class 1 or 2), a detection antibody (selected from the opposite class: class 1 or 2), and an enzyme-linked anti-human antibody (i.e., an antibody with a detectable marker) which binds to the detection antibody.

In some embodiments, the methods and systems described herein can be used to sequence antibodies against the SARS-CoV-2 spike protein. In such embodiments, antibodies against the spike protein can be used to develop a sandwich ELISA method as described herein to detect a SARS-CoV-2 infection in a subject. In some embodiments, the methods and systems described herein can also be used to sequence and synthesize neutralizing antibodies against SARS-CoV-2 NP and spike protein.

In some embodiments, the invention provides for a nucleic acid encoding the any of the antibodies described above. In some embodiments, the nucleic acid is operably linked to a promoter inserted in an expression vector. In some embodiments, the expression vector is a bacterial expression vector.

### REFERENCES

Ankur Garg, Lihong Liu, David D. Ho, and Leemor Joshua-Tor. (2020). Heterologous Expression and Purification of SARS-CoV2 Nucleocapsid Protein. Bio-101: e5005. DOI: 10.21769/BioProtoc.5005.
Pengfei Wang, Lihong Liu, Manoj S. Nair, Michael T. Yin, Yang Luo, Qian Wang, Ting Yuan, Kanako Mori, Axel Guzman Solis, Masahiro Yamashita, Lawrence J. Purpura, Justin C. Laracy, Jian Yu, Joseph Sodroski, Yaoxing Huang, David D. Ho, (2020). SARS-CoV-2 Neutralizing Antibody Responses Are More Robust in Patients with Severe Disease, doi: https://doi.org/10.1101/2020.06.13.150250.
Lihong Liu, Pengfei Wang, Manoj S. Nair, Jian Yu, Yaoxing Huang, Micah A. Rapp, Qian Wang, Yang Luo, Vincent Sahi, Amir Figueroa, Xinzheng V. Guo, Gabriele Cerutti, Jude Bimela, Jason Gorman, Tongqing Zhou, Peter D. Kwong, Joseph G. Sodroski, Michael T. Yin, Zizhang Sheng, Lawrence Shapiro, David D. Ho, (2020). Potent Neutralizing Monoclonal Antibodies Directed to Multiple Epitopes on the SARS-CoV-2 Spike. Doi: https://doi.org/10.1101/2020.06.17.153486

The devices, systems, and methods disclosed herein are not to be limited in scope to the specific embodiments described herein. Indeed, various modifications of the devices, systems, and methods in addition to those described will become apparent to those of skill in the art from the foregoing description.

1. A kit for detecting or quantifying a SARS-CoV-2 nucleocapsid phosphoprotein, the kit comprising:
a first antibody, wherein a variable heavy chain domain of the first antibody comprises an amino acid sequence selected from the group consisting of SEQ ID NO: 1, SEQ ID NO: 3, SEQ ID NO: 5, SEQ ID NO: 7, SEQ ID NO: 11, SEQ ID NO: 13, SEQ ID NO: 15, SEQ ID NO: 17, SEQ ID NO: 19, and SEQ ID NO: 21, and wherein a variable light chain domain of the first antibody comprises an amino acid sequence selected from the group consisting of SEQ ID NO: 2, SEQ ID NO: 4, SEQ ID NO: 6, SEQ ID NO: 8, SEQ ID NO: 12, SEQ ID NO: 14, SEQ ID NO: 16, SEQ ID NO: 18, SEQ ID NO: 20, and SEQ ID NO: 22; and
a second antibody.

2. The kit of claim 1, further comprising an ELISA plate having a plurality of compartments.

3. The kit of claim 2, wherein the plurality of compartments comprises a reaction chamber and wherein one or more of the plurality of compartments further comprises one of the first antibody or the second antibody.

4. The kit of claim 1, further comprising a third antibody, wherein the third antibody is an anti-human antibody comprising a detectable marker.

5. The kit of claim 1, further comprising an enzyme linked to one of the first antibody or the second antibody.

6. The kit of claim 4, further comprising a substrate capable of detecting the detectable marker.

7. The kit of claim 1, wherein the first antibody is a capture antibody and the second antibody is a detection antibody.

8. The kit of claim 1, wherein the second antibody is a capture antibody and the first antibody is a detection antibody.

9. The kit of claim 1, wherein the variable heavy chain domain of the first antibody comprises the amino acid sequence of SEQ ID NO: 1 and the variable light chain domain of the first antibody comprises the amino acid sequence of SEQ ID NO: 2.

10. The kit of claim 1, wherein the variable heavy chain domain of the first antibody comprises the amino acid sequence of SEQ ID NO: 3 and the variable light chain domain of the first antibody comprises the amino acid sequence of SEQ ID NO: 4.

11. The kit of claim 1, wherein the variable heavy chain domain of the first antibody comprises the amino acid sequence of SEQ ID NO: 5 and the variable light chain domain of the first antibody comprises the amino acid sequence of SEQ ID NO: 6.

12. The kit of claim 1, wherein the variable heavy chain domain of the first antibody comprises the amino acid sequence of SEQ ID NO: 7 and the variable light chain domain of the first antibody comprises the amino acid sequence of SEQ ID NO: 8.

13. The kit of claim 1, wherein the variable heavy chain domain of the first antibody comprises the amino acid sequence of SEQ ID NO: 11 and the variable light chain domain of the first antibody comprises the amino acid sequence of SEQ ID NO: 12.

14. The kit of claim 1, wherein the variable heavy chain domain of the first antibody comprises the amino acid sequence of SEQ ID NO: 13 and the variable light chain domain of the first antibody comprises the amino acid sequence of SEQ ID NO: 14.

15. The kit of claim 1, wherein the variable heavy chain domain of the first antibody comprises the amino acid sequence of SEQ ID NO: 15 and the variable light chain domain of the first antibody comprises the amino acid sequence of SEQ ID NO: 16.

16. The kit of claim 1, wherein the variable heavy chain domain of the first antibody comprises the amino acid sequence of SEQ ID NO: 17 and the variable light chain domain of the first antibody comprises the amino acid sequence of SEQ ID NO: 18.

17. The kit of claim 1, wherein the variable heavy chain domain of the first antibody comprises the amino acid sequence of SEQ ID NO: 19 and the variable light chain domain of the first antibody comprises the amino acid sequence of SEQ ID NO: 20.

18. The kit of claim 1, wherein the variable heavy chain domain of the first antibody comprises the amino acid sequence of SEQ ID NO: 21 and the variable light chain domain of the first antibody comprises the amino acid sequence of SEQ ID NO: 22.

19. The kit of claim 1, wherein the variable heavy chain domain of the second antibody comprises the amino acid sequence of SEQ ID NO: 9 and the variable light chain domain of the second antibody comprises the amino acid sequence of SEQ ID NO: 10.

20. The kit of claim 1, wherein the variable heavy chain domain of the second antibody comprises the amino acid sequence of SEQ ID NO: 13 and the variable light chain domain of the second antibody comprises the amino acid sequence of SEQ ID NO: 14.

21. The kit of claim 1, wherein each of the first antibody and the second antibody bind to different epitopes of the SARS-CoV-2 nucleocapsid phosphoprotein.

22. The kit of claim 1, wherein the kit is configured to detect, in a biological sample, the presence of a SARS-CoV-2 nucleocapsid phosphoprotein.

23-54. (canceled)

55. The kit of claim 1, wherein the first antibody or the second antibody comprises a detectable marker, and wherein the kit further comprises a substrate capable of detecting the detectable marker.

56. The kit of claim 1, wherein a variable heavy chain domain of the second antibody comprises an amino acid sequence selected from the group consisting of SEQ ID NO: 9, and SEQ ID NO: 13, and wherein a variable light chain domain of the second antibody comprises an amino acid sequence selected from the group consisting of SEQ ID NO: 10, and SEQ ID NO: 14.


SEQUENCE LISTING (22 sequences)

SEQ ID NO: 1 - protein, 122 aa, Artificial Sequence
  Description of Artificial Sequence Synthetic polypeptide
QVQLVQSGAEVKKPGASVKVSCKASGYTFTSYAISWVRQAPGQGLEWMGWISAYTGNTNYAQKLQGRVTMTTDTSTSTAYMELRSLRSDDTAVYYCARNGWDYDTSGTHDYWGQGTLVTVSS

SEQ ID NO: 2 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 3 - protein, 127 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITAEESTSTAYMELSSLRSEDTAVYYCARDGWAAAGPDTSLLGTFDIWGQGTMVTVSS

SEQ ID NO: 4 - protein, 111 aa, Artificial Sequence
  Description of Artificial Sequence Synthetic polypeptide
QSALTQPPSVSGSPGQSVTISCTGTSSDVGSYNRVSWYQQPPGTAPKLIIYEVSNRPSGVPDRFSGSKSGNTASLTISGLQAEDEADYYCSSYTSSSTYVVFGGGTKLTVL

SEQ ID NO: 5 - protein, 128 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTSWGSGSYYKTYYYNGMDVWGQGTTGTVSS

SEQ ID NO: 6 - protein, 112 aa, Artificial Sequence
  Description of Artificial Sequence Synthetic polypeptide
QSVLTQPPSASGTPGQRVTISCSGSSSNIGSNTVNWYQQLPGTAPKLLIYTNNQRPSGVPDRFSGSKSGTSASLAISGLQSEDEADYYCAAWDDSLNGRYVVFGGGTKLTVL

SEQ ID NO: 7 - protein, 129 aa, Artificial Sequence
  Description of Artificial Sequence Synthetic polypeptide
QVQLVQSGAEVKKPGSSVKVSCKASGGTFSNYVISWVRQAPGQGLEWMGGIIPIFGTAKYAQKFQGRVAITADESTSTAYMEVSSLRSEDTAVYYCARAGYCSGGSCRRPSDYYGMDVWGQGTTVTVSS

SEQ ID NO: 8 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKLEIK

SEQ ID NO: 9 - protein, 115 aa, Artificial Sequence
  Description of Artificial Sequence Synthetic polypeptide
EVQLVESGGGLVQPGGSLRLSCAASGFTFSDSAVHWVRQASGKGLEWVGRIRNRNNNYATAYAASVKGRFTISRDDSENMAYLQMNGLKTEDTAIYYCTDLLAYWGQGTLLTVSS

SEQ ID NO: 10 - protein, 108 aa, Artificial Sequence
  Description of Artificial Sequence Synthetic polypeptide
SYELTQPPSVSVSPGQTARITCSADALPKQYAYWYQQKAGQAPVLVIYKDNERPSGIPERFSGSSSGTTVTLTISGVQAEDEADYYCQSADSSGGYRVFGGGTKLTVL

SEQ ID NO: 11 - protein, 123 aa, Artificial Sequence
  Description of Artificial Sequence Synthetic polypeptide
EVQLVESGGGLVQPGGSLRLSCAASGFTFSTYWMSWVRQAPGKGLEWVANIKQDGSEKYYVDSVKGRFTISRDNAKNSLYLQMNSLRAEDTAVYYCARYSLDYYDTSGSFDYWGQGTLVAVSS

SEQ ID NO: 12 - protein, 109 aa, Artificial Sequence
  Description of Artificial Sequence Synthetic polypeptide
SYELTQPPSVSVSPGQTARITCSGDALPKKYAYWYQQKSGQAPVLVIYEDSKRPSGIPERFSGSSSGTMATLTISGAQVEDEADYYCYSTDSSGNHRGVFGGGTQLTVL

SEQ ID NO: 13 - protein, 131 aa, Artificial Sequence
  Description of Artificial Sequence Synthetic polypeptide
EVQLVESGGGLVQPGGSLRLSCAASGFIFSNYWMSWVRQAPGKGLEWVANTKQDDSEKYYVDAVKGRFTISRDNAKNSLYLQMNSLRADDTAVYYCAREVRIAVTGTSRDEDYSYNGMDVWGQGTTVTVSS

SEQ ID NO: 14 - protein, 108 aa, Artificial Sequence
  Description of Artificial Sequence Synthetic polypeptide
SYELTQPPSVSVSPGQTARITCSADALAKQYAYWYQQKPGQAPVLVIFKDSERPSGIPERFSGSSSGTTVTLTISRVQAEDEADYYCQSADSSGYYWAFGGGTKLTVL

SEQ ID NO: 15 - protein, 120 aa, Artificial Sequence
  Description of Artificial Sequence Synthetic polypeptide
EVQLVQSGAEVKKSGESLKISCKGSGYSFTNYWIGWVRQMPGKGLEWMGIIYPGDSDTRHSPSFQGQVTISADKSLRTAYLQWSSLKASDTAIYYCARGADGYSSYFDYWGQGTLVTVSS

SEQ ID NO: 16 - protein, 110 aa, Artificial Sequence
  Description of Artificial Sequence Synthetic polypeptide
NFMLTQPHSVSESPGKTVIISCTRSSGSIASDYVQWYQQRPGSVPTTVIYEDNERPSGVPDRFSGSIDSSSNSASLTISGLKTEDEADYYCQSYDSSVQVFGGGTKLTVL

SEQ ID NO: 17 - protein, 452 aa, Artificial Sequence
  Description of Artificial Sequence Synthetic polypeptide
QVQLVQSGAEVKKPGASVKVSCKASGYTFTSYAISWVRQAPGQGLEWMGWISAYTGNTNYAQKLQGRVTMTTDTSTSTAYMELRSLRSDDTAVYYCARNGWDYDTSGTHDYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 18 - protein, 215 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 457 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITAEESTSTAYMELSSLRSEDTAVYYCARDGWAAAGPDTSLLGTFDIWGQGTMVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 20 - protein, 216 aa, Artificial Sequence
  Description of Artificial Sequence Synthetic polypeptide
QSALTQPPSVSGSPGQSVTISCTGTSSDVGSYNRVSWYQQPPGTAPKLIIYEVSNRPSGVPDRFSGSKSGNTASLTISGLQAEDEADYYCSSYTSSSTYVVFGGGTKLTVLGQPKAAPSVTLFPPSSEELKENKATLVCLISNFSPSGVTVAWKANGTPITQGVDTSNPTKEGNKFMASSFLHLTSDQWRSHNSFTCQVTHEGDTVEKSLSPAECL

SEQ ID NO: 21 - protein, 458 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTSWGSGSYYKTYYYNGMDVWGQGTTGTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 217 aa, Artificial Sequence
  Description of Artificial Sequence Synthetic polypeptide
QSVLTQPPSASGTPGQRVTISCSGSSSNIGSNTVNWYQQLPGTAPKLLIYTNNQRPSGVPDRFSGSKSGTSASLAISGLQSEDEADYYCAAWDDSLNGRYVVFGGGTKLTVLGQPKAAPSVTLFPPSSEELKENKATLVCLISNFSPSGVTVAWKANGTPITQGVDTSNPTKEGNKFMASSFLHLTSDQWRSHNSFTCQVTHEGDTVEKSLSPAECL